(19) United States
(12) Patent Application Publication
(10) Pub. No.: US 2020/0155830 A1
(43) Pub. Date: May 21, 2020

(54) NASAL NEUROSTIMULATION DEVICE WITH ELECTRICALLY CONDUCTIVE PLASTIC ELECTRODE

(71) Applicant: ALLERGAN / Oculeve, Madison, NJ (US)

(72) Inventors: Jarren A. Baldwin, Oakland, CA (US); Kaustubh Chitre, Irvine, CA (US); Allen Fung, Santa Clara, CA (US); Chao Liu, San Francisco, CA (US); John Wardle, San Clemente, CA (US)

(21) Appl. No.: 16/685,980

(22) Filed: Nov. 15, 2019

Related U.S. Application Data
(60) Provisional application No. 62/768,584, filed on Nov. 16, 2018.

(51) Int. Cl. A61N 1/05; A61N 1/36; A61N 1/04

(52) U.S. Cl. CPC A61N 1/0546; A61N 1/0456; A61N 1/36046

(57) ABSTRACT

Various embodiments of a nasal stimulator probe is described that is configured to assist with providing a stimulation to nasal tissue of a subject. In some embodiments, the nasal stimulator probe is configured to releasably couple to a stimulator body including a power source. The stimulator probe may include a first extension of a first nasal insertion prong configured for insertion into a nasal cavity. Additionally, the stimulator probe may include a first electrode configured to provide the stimulation and coupled to a distal end of the first extension. In addition, the first electrode may include a conductive plastic material. Systems and method associated with the nasal stimulator probe are also described.

### CROSS-REFERENCE TO RELATED APPLICATION

The current application claims priority under 35 U.S.C. § 119(e) to U.S. Provisional patent application Ser. No. 62/768,584, filed on Nov. 16, 2018 and entitled “Nasal Neurostimulation Device With Electrically Conductive Plastic Electrode,” which is incorporated by reference herein in its entirety.

### TECHNICAL FIELD

The subject matter described herein relates to a handheld nasal stimulator and related methods of use.

### BACKGROUND

Dry Eye Disease (“DED”) is a condition that affects millions of people worldwide. More than 40 million people in North America have some form of dry eye, and many millions more suffer worldwide. DED results from the disruption of the natural tear film on the surface of the eye, and can result in ocular discomfort, visual disturbance and a reduction in vision-related quality of life. Activities of daily living such as driving, computer use, housework and reading have also been shown to be negatively impacted by DED. Patients with severe cases of DED are at risk for serious ocular health deficiencies such as corneal ulceration, and can experience a quality of life deficiency comparable to that of moderate-severe angina.

The etiology of DED is becoming increasingly well understood. DED is progressive in nature, and fundamentally results from insufficient tear coverage on the surface of the eye. This poor tear coverage prevents healthy gas exchange and nutrient transport for the ocular surface, promotes cellular desiccation and creates a poor refractive surface for vision. Poor tear coverage typically results from: 1) insufficient aqueous tear production from the lacrimal glands (e.g. secondary to post-menopausal hormonal deficiency, auto-immune disease, LASIK surgery, etc.), and/or 2) excessive evaporation of aqueous tear resulting from dysfunction of the meibomian glands. Low tear volume causes a hyperosmolar environment that induces an inflamed state of the ocular surface. This inflammatory response induces apoptosis of the surface cells which in turn prevents proper distribution of the tear film on the ocular surface so that any given tear volume is rendered less effective. This initiates a vicious cycle where more inflammation can ensue causing more surface cell damage, etc. Additionally, the neural control loop, which controls reflex tear activation, is disrupted because the sensory neurons in the surface of the eye are damaged. As a result, fewer tears are secreted and a second vicious cycle develops that results in further progression of the disease (fewer tears cause nerve cell loss, which results in fewer tears, etc.). Accordingly, effective treatment for dry eye is desired.

### SUMMARY

Aspects of the current subject matter include various embodiments of a handheld stimulator system configured to provide a stimulation to a subject. The handheld stimulator system may include a nasal stimulator probe and a stimulator body. In one aspect, an embodiment of a nasal stimulator probe is configured to releasably couple to a stimulator body for providing a stimulation to nasal tissue of a subject. The stimulator probe may include a first extension of a first nasal insertion prong configured for insertion into a nasal cavity. Additionally, the stimulator probe may include a first electrode configured to provide the stimulation and coupled to a distal end of the first extension. In addition, the first electrode may include a conductive plastic material.

In some variations one or more of the following features can optionally be included in any feasible combination. In some embodiments, the conductive plastic material may include a carbon black material. In some embodiments, the conductive plastic material may include one or more of a graphene material, carbon fibers, and a metal polymer. The conductive plastic material may assist with providing a conductive pathway between a power source in the stimulator body and nasal tissue. The first electrode may include a shape having an arc of a cylindrical surface. The first electrode may include an outer contact wall including a radius of approximately 3 mm to approximately 7 mm.

In some embodiments, at least a part of the first electrode may be covered with a biocompatible conductive coating and/or a titanium material. In some embodiments, the conductive plastic material may include one or more of a polyethylene material, an ethylene vinyl acetate material, and a polypropylene material. In some embodiments, the conductive plastic material of the first electrode may include a volume comprising approximately 3% to approximately 30% carbon black filler.

In some embodiments, the first electrode may be in electrical communication with a power source positioned in the stimulator body when the nasal insertion probe is coupled to the stimulator body. In some embodiments, the nasal insertion probe may further include a second extension of a second nasal insertion prong configured for insertion into a nasal cavity, as well as a second electrode coupled to a distal end of the second extension and including the conductive plastic material.

In another aspect of the current subject matter, an embodiment of a handheld stimulator system is configured to provide a stimulation to nasal tissue of a subject and includes a stimulator body including a power source. The handheld stimulator system may further include a nasal stimulator probe configured to releasably couple to the stimulator body. The nasal stimulator probe may include a first extension of a first nasal insertion prong configured for insertion into a nasal cavity, as well as a first electrode configured to provide the stimulation and coupled to a distal end of the first extension. The first electrode may include a conductive plastic material

In another interrelated aspect of the current subject matter, a method includes delivering, via a conductive plastic material of a nasal stimulator probe of a handheld stimulator system, a stimulation to a nasal tissue of a subject. The nasal stimulator probe may include a first extension of a first nasal insertion prong configured for insertion into a nasal cavity, as well as a first electrode configured to provide the stimulation and coupled to a distal end of the first extension, the first electrode including a conductive plastic material. In some embodiments, the method can further include releasably coupling the nasal insertion probe to a stimulator body including a power source.

The details of one or more variations of the subject matter described herein are set forth in the accompanying drawings and the description below. Other features and advantages of the subject matter described herein will be apparent from the description and drawings, and from the claims.

### DESCRIPTION OF DRAWINGS

The accompanying drawings, which are incorporated in and constitute a part of this specification, show certain aspects of the subject matter disclosed herein and, together with the description, help explain some of the principles associated with the disclosed implementations. In the drawings,

FIGS. 1A, 1B, 1C, 1D, 1E show perspective, front, back, cut-away back, and cut-away side views, respectively, of an illustrative embodiment of a handheld stimulator;

FIG. 1F depicts a cut-away back view of a stimulator probe of the handheld stimulator of FIG. 1A;

FIG. 2 shows a block diagram schematically representing a variation of a stimulator; and

FIG. 3 depicts a partially exploded perspective view of the stimulator probe of FIG. 1F including electrodes made out of a conductive plastic material.

When practical, similar reference numbers denote similar structures, features, or elements.

### DETAILED DESCRIPTION

This disclosure describes devices, systems, and methods for treating one or more conditions (such as dry eye) by providing stimulation to nasal or sinus tissue. The devices and systems may be configured to stimulate nasal or sinus tissue. In some variations, the devices may comprise a stimulator body and a stimulator probe, where the stimulator probe comprises one or more nasal insertion prongs. The stimulus delivered by the stimulators described herein may be electrical. When the devices and systems are used to treat dry eye, the methods may comprise stimulating nasal or sinus tissue to increase tear production, reduce the symptoms of dry eye, or improve ocular appearance and/or health.

Furthermore, embodiments of a stimulator are described that include conductive plastic electrodes. Such conductive plastic electrodes may be formed in a variety of shapes and sizes and configured to provide a desired resistance. As will be described in greater detail below, some embodiments of the conductive plastic electrodes may include a carbon black material that is added to a plastic material to thereby make the plastic material conductive. The conductive plastic electrodes may provide a variety of benefits, including improved cost and efficiency related to the manufacturing and assembly of the stimulator. Other benefits are also within the scope of this disclosure.

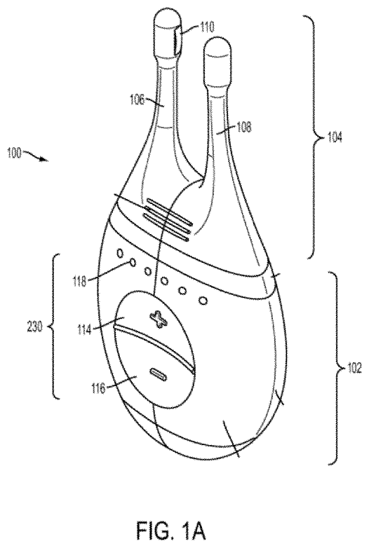

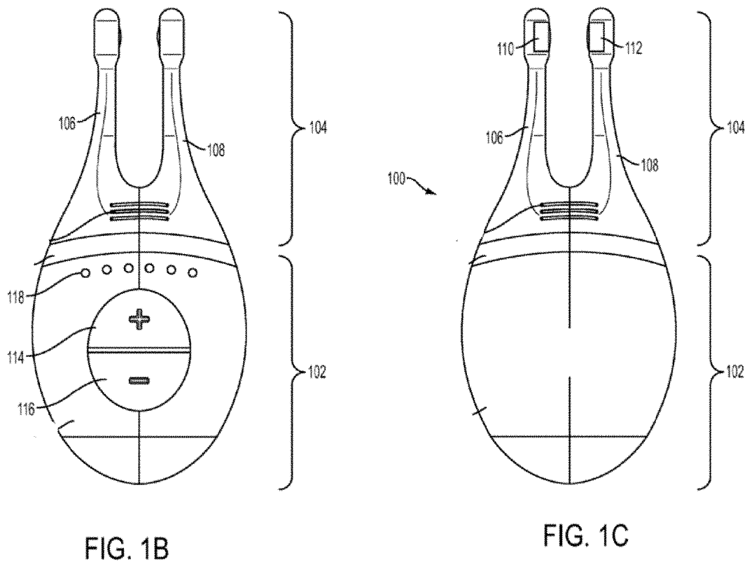

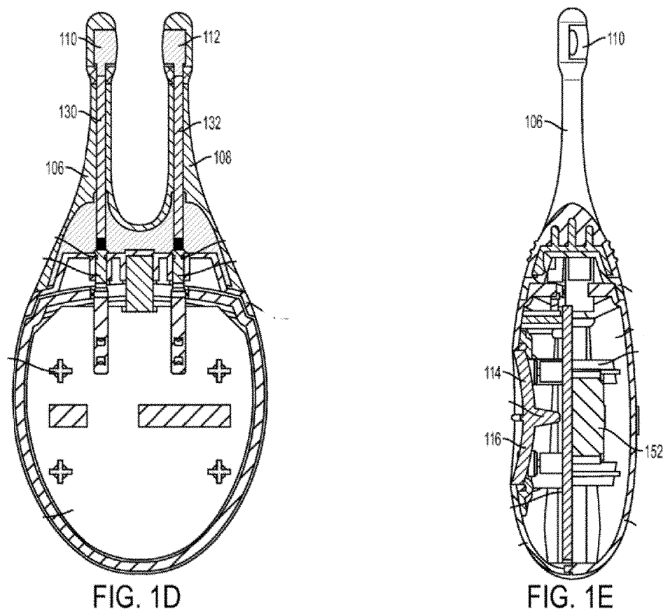

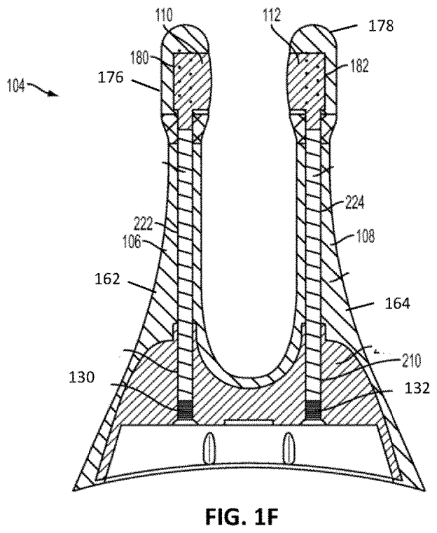

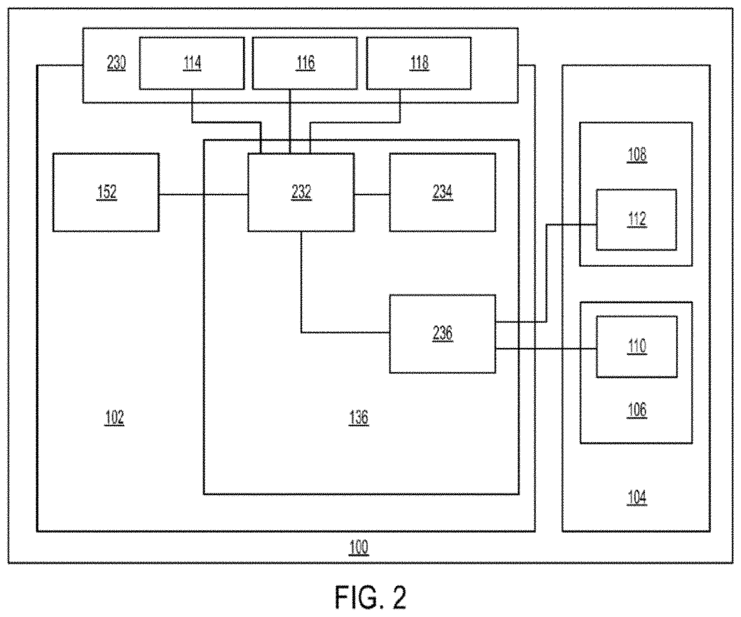

Some variations of the stimulation systems described here may comprise a handheld stimulator. FIGS. 1A, 1B, 1C, 1D, 1E show perspective, front, back, cut-away back, and cut-away side views, respectively, of an illustrative variation of a handheld stimulator 100, respectively. FIG. 1F depicts a cut-away back view of a stimulator probe of the handheld stimulator shown in FIG. 1A. FIG. 2 shows a block diagram schematically representing the stimulator 100. As shown in FIGS. 1A-1E, the stimulator 100 may comprise a stimulator body 102 and a stimulator probe 104. The stimulator body 102 may be configured to generate a stimulus that may be delivered to a subject. The stimulator body 102 may contain a control subsystem 136 and a power source 152, which together may generate and control the stimulus. The stimulator probe 104 of the stimulator may comprise one or more nasal insertion prongs 106 and 108, which may be configured to extend at least partially into a nasal cavity of a subject.

The stimulus may be delivered to a subject via the stimulator probe 104. In some variations, the stimulator body 102 and stimulator probe 104 may be reversibly attachable. In other variations, the stimulator probe may be permanently connected to the stimulator body. Some or all of the stimulator 100 may be disposable. In other variations, one or more portions of the stimulator 100 may be reusable. For example, in variations where the stimulator probe 104 is releasably connected to the stimulator body 102, the stimulator body 102 may be reusable, and the stimulator probe 104 may be disposable and periodically replaced.

In some variations, the stimulus may be electrical. In these instances, each nasal insertion prong may comprise at least one electrode. As shown, the stimulator probe 104 may comprise a first electrode 110 on nasal insertion prong 106 and a second electrode 112 on nasal insertion prong 108. As shown in the cut-away view of the stimulator 100 in FIG. 1F, the electrodes 110 and 112 may be connected to leads 130 and 132 located within prongs 106 and 108, respectively. The leads 130 and 132 may connect directly or indirectly to the control subsystem 136 and power source 152. As such, the electrical stimulus may travel from the control subsystem 136, through the leads 130 and 132, and through the electrodes 110 and 112 to thereby provide a stimulus to tissue of a subject (e.g., facial tissue, tissue of the nasal cavity, etc.).

The power source may be any suitable power supply capable of powering one or more functions of the stimulator, such as one or more batteries, capacitors, or the like. In some embodiments, the stimulator body 102 comprises a power source, in other variations the stimulator body 102 need not comprise a power source. In some variations, the stimulator body may comprise a port, cord, or other mechanism for connecting the stimulator to an external power source (such as a wall outlet or separate battery pack), which in turn may be used to power one or more portions of the stimulator.

As shown in FIGS. 1A and 2, the stimulator body 102 may comprise a user interface 230 comprising one or more operating mechanisms to adjust one or more parameters of the stimulus. The operating mechanisms may provide information to the control subsystem 136, which may comprise a processor 232, memory 234, and/or stimulation subsystem 236. In some variations, the operating mechanisms may comprise first and second buttons 114 and 116. In some variations, pressing the first button 114 may turn on the stimulator and/or change one or more parameters of the stimulus (e.g., increase the intensity of the stimulus, change the stimulation pattern, or the like), while pressing the second button 116 may turn off the stimulator and/or change one or more parameters of the stimulus (e.g., decrease the intensity of the stimulus, change the stimulation pattern, or the like). Additionally or alternatively, the user interface may comprise one or more feedback elements (e.g., based on light, sound, vibration, or the like). As shown, the user feedback elements may comprise light-based indicators 118, which may provide information to the user, as described in more detail below.

As discussed above, the nasal insertion prongs 106 and 108 may be configured to be inserted in a subject's nostrils, however, the nasal insertion prongs 106 and 108 may be configured for stimulating other facial tissue. As shown in FIG. 1F, each nasal insertion prong 106 and 108 may comprise an elongate portion 162 and 164, respectively. Each elongate portion 162 and 164 may have at its distal end a distal portion 176 and 178. In some variations, the distal portions 176 and 178 may have a diameter (or greatest cross-sectional dimension) that is larger than the diameter (or greatest cross-sectional dimension) of the elongate portion 162 and 164 of the prongs proximal to the distal portions. This may allow a portion of the distal portions 176 and/or 178 (e.g., the electrodes, described below) to be brought into contact with a subject's tissue, while the elongate portions 162 and 164 are not in contact with the subject's tissue. For example, the diameter of the nasal insertion prongs 106 and 108 at the distal portions 176 and 178 may in some instances be between about 3 mm and about 7 mm, while the diameter of the elongate portions 162 and 164 may be between about 1 mm and about 6 mm proximal to the distal portions. More specifically, in some variations the diameter of the nasal insertion prongs at the distal portions 176 and 178 may be about 5 mm, and the diameter of the elongate portions 162 and 164 may be about 3 mm.

When the stimulators described here are configured to deliver an electrical stimulus, at least one of the nasal insertion prongs may comprise one or more electrodes configured to deliver a stimulus to tissue. In variations where a stimulator comprises two nasal insertion prongs, each of the two nasal insertion prongs may comprise at least one electrode. For example, having multiple electrode-bearing prongs may allow the stimulator to provide bipolar stimulation (and/or bilateral stimulation of two nostrils).

Various embodiments of electrodes are described herein. In some embodiments, the electrode is made from one or more conductive materials. In some variations, the electrode may comprise one or more materials configured to promote electrical contact between electrodes of the stimulator probe and tissue (i.e., all of an electrodes or a portion of the electrode, such as a covering). In some instances, the impedance provided by tissue may be at least partially dependent on the presence or absence of fluid-like materials (e.g., mucous) in the nasal cavity. The material(s) may help to minimize the impact of subject tissue impedance by providing a wet interface between the electrode and tissue, which may act to normalize the impedance experienced by the electrodes. This may in turn normalize the output and sensation experienced by the user.

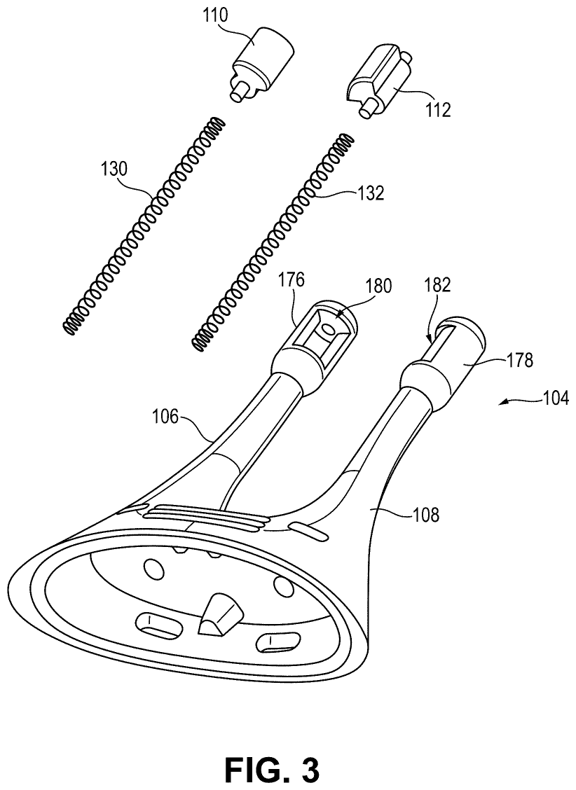

As shown in FIG. 3, the electrodes 110 and 112 may be formed of a plastic material loaded with a carbon black filler material. The carbon black filler may be conductive and allow current to travel between particles of carbon black of the carbon black filler contained or suspended within the plastic material. As such, the carbon black filler may act as a micro-circuit. The plastic material may include insulative properties and include one or more of a variety of plastic materials such as Polyethylene (PE), Ethylene vinyl acetate (EVA), Polypropylene (PP), etc. The ratio of carbon black filler volume to plastic material volume may affect the resistance properties of the conductive plastic electrode. For example, the percent of volume of carbon black filler of the conductive plastic material forming the electrode may be within a range of approximately 3% to approximately 30%. The carbon black filler may include carbon black pellets that may vary in size and/or shape. In some embodiments, the conductive plastic material may include one or more of a graphene material, carbon fibers, and a metal polymer, such as instead of or in addition to the carbon black filler.

In some implementations, during manufacturing of the conductive plastic electrode, the carbon black filler may be uniformly dispersed using a melting and mixing process. Furthermore, the conductive plastic electrode may be formed using any one of a variety of molding techniques.

In some variations, at least one conductive plastic electrode may further be covered with a biocompatible conductive coating, such as along a surface of the conductive plastic electrode that is configured to contact a tissue surface. Such a conductive coating may allow current and/or stimulation to pass therethrough while providing a protective barrier between the user and the conductive plastic electrode. For example, the biocompatible conductive coating may be made out of a titanium material, however, other materials are within the scope of this disclosure.

As shown in FIG. 3, the electrodes 110 and 112 may be made out of a variation of conductive plastic material described herein and configured to couple to the openings 180 and 182 of the distal portions 176 and 178 of the nasal insertion prongs 106 and 108. For example, the conductive plastic electrodes 110 and 112 may form about a 100 degree arc of a cylinder, although it should be appreciated that the conductive plastic electrodes 110 and 112 may in other variations have other shapes (e.g., a smaller or larger arc, as described in detail herein). In some embodiments, the electrodes 110, 112 may include an outer contact wall (e.g., configured to contact nasal tissue) including a radius of approximately 3 mm to approximately 7 mm. The conductive plastic electrodes may each substantially fill the respective openings 180 and 182 when coupled thereto and contact leads 130 and 132 located adjacent to the openings 180 and 182.

In variations in which the electrodes 110 and 112 comprise an arc of a cylindrical surface, such as in the variation shown in FIG. 3, the electrodes 110 and 112 may comprise about a 100 degree arc of a cylindrical surface. That is, openings 180 and 182 in the distal portions 176 and 178 of the nasal insertion prongs may comprise about a 100 degree arc of a cylinder, and the electrodes 110 and 112 may be located within the openings 180 and 182. In other variations, the electrodes may be any suitable arc length of a cylinder. For example, in some embodiments, the electrodes may be semi-cylindrical. In other embodiments, the electrodes may be a partial cylinder having an arc greater than 100 degrees (e.g., between about 110 degrees and about 270 degrees, about 110 degrees, about 120 degrees, about 180 degrees, about 270 degrees, or the like). In yet other embodiments, an electrode may be a partial cylinder having an arc less than 100 degrees (e.g., between about 30 degrees about 95 degrees, about 90 degrees, about 45 degrees, or the like). Additionally, some embodiments of the electrodes 110 and 112 may have an outer surface radius that is the same as or greater than an outer surface radius of the distal portions 176 and 178. For example, the electrodes 110 and 112 may each have a greater radius compared to their respective distal portion 176 and 178 such that the outer surface (e.g., outer contact wall) of the electrode extends away from and/or protrudes from the outer surface of the distal portions 176 and 178. This can assist with ensuring the electrodes 110 and 112 make sufficient contact with tissue for allowing a stimulus to be delivered to the tissue.

Although the electrodes 110 and 112 described above may comprise an arc of a cylindrical surface, it should be appreciated that the electrodes described herein may have any suitable shape. In some other variations, for example, the electrodes 110 and 112 may comprise two or more adjacent arcs of a cylindrical surface. For example, the nasal insertion prongs 106 and 108 may comprise two semi-cylindrical electrodes. In yet other variations, the electrodes 110 and 112 may comprise a portion of an arc of a cylindrical surface, wherein the portion of the arc comprises rounded edges. In some other variations, for example, an electrode may be ellipsoid or spherical, ovoid, or the like. In yet other variations, the electrodes may comprise an array of electrodes. In some variations, having an array of electrodes may allow a stimulus to be delivered to tissue even if one or more of the electrodes in the array fails, and/or may facilitate unilateral stimulation with a single nasal insertion prong.

In some variations, the center of the electrodes 110 and 112 may face each other. In some variations, the center of the electrodes may be positioned at an angle relative to each other. As such, for example, when the stimulator probe 104 is positioned such that the first nasal insertion prong is positioned in a first nostril and the second nasal insertion prong is positioned in the second nostril, the electrodes 110 and 112 may be directed partially toward the front of the nose.

The electrodes 110 and 112 may be positioned on any suitable longitudinal portion or portions of the nasal insertion prongs. The position of the electrode along the prong may at least partially determine the placement of the electrode relative to tissue when the stimulator probe is advanced into the nose. In some variations, the electrodes may be positioned such that when inserted into the nasal cavity, the electrodes are capable of reaching the nasal mucosa or other area desired to be stimulated.

When a nasal insertion prong or prongs of the stimulators described herein comprise one or more electrodes, the electrodes may comprise leads. When the stimulator probe is connected to a stimulator body, the leads may contact the circuitry of the stimulator body to electrically connect the electrodes to the stimulator body circuitry, as described in more detail below. As such, the leads may extend at least partially through each of the nasal insertion prongs. The leads may be formed from one or more conductive materials (e.g., stainless steel, titanium, platinum or platinum-iridium, other alloys thereof, or the like), conductive ceramics (e.g., titanium nitride), and may be positioned such that at least a portion of each lead contacts a respective electrode to provide a conduction pathway between the lead and the electrode.

For example, as shown in FIGS. 1F and 3, the leads 130 and 132 may each comprise a spring. The spring leads 130 and 132 may comprise any suitable biocompatible conductive material or materials. For example, in some variations, the springs may comprise stainless steel. In other variations, the springs may comprise gold or platinum. In some variations, the springs may comprise two or more materials (e.g., stainless steel with gold plating). The leads 130 and 132 may extend through the central lumens 222 and 224 of the nasal insertion prongs 106 and 108, respectively. A portion of the leads (e.g., the distal ends) may contact the electrodes 110 and 112. The proximal ends of the leads 130 and 132 may contact the circuitry of the stimulator body.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

The implementations set forth in the foregoing description do not represent all implementations consistent with the subject matter described herein. Instead, they are merely some examples consistent with aspects related to the described subject matter. Although a few variations have been described in detail herein, other modifications or additions are possible. In particular, further features and/or variations can be provided in addition to those set forth herein. For example, the implementations described above can be directed to various combinations and sub-combinations of the disclosed features and/or combinations and sub-combinations of one or more features further to those disclosed herein. In addition, the logic flows depicted in the accompanying figures and/or described herein do not necessarily require the particular order shown, or sequential order, to achieve desirable results. The scope of the following claims may include other implementations or embodiments.

What is claimed is:

1. A nasal stimulator probe configured to releasably couple to a stimulator body for providing a stimulation to nasal tissue of a subject, the stimulator probe comprising:
a first extension of a first nasal insertion prong configured for insertion into a nasal cavity; and
a first electrode coupled to a distal end of the first extension, the first electrode including a conductive plastic material and configured to provide the stimulation to nasal tissue.

2. The nasal stimulator probe of claim 1, wherein the conductive plastic material includes a carbon black material.

3. The nasal insertion probe of claim 1, wherein the conductive plastic material includes one or more of a graphene material, carbon fibers, and a metal polymer.

4. The nasal insertion probe of claim 1, wherein the conductive plastic material assists with providing a conductive pathway between a power source in the stimulator body and nasal tissue.

5. The nasal insertion probe of claim 1, wherein the first electrode includes a shape comprising an arc of a cylindrical surface.

6. The nasal insertion probe of claim 1, wherein the first electrode includes an outer contact wall including a radius of approximately 3 mm to approximately 7 mm.

7. The nasal insertion probe of claim 1, wherein at least a part of the first electrode is covered with a biocompatible conductive coating and/or a titanium material.

8. The nasal insertion probe of claim 1, wherein the conductive plastic material includes one or more of a polyethylene material, an ethylene vinyl acetate material, and a polypropylene material.

9. The nasal insertion probe of claim 2, wherein the conductive plastic material of the first electrode includes a volume comprising approximately 3% to approximately 30% carbon black filler.

10. The nasal insertion probe of claim 1, wherein the first electrode is in electrical communication with a power source positioned in the stimulator body when the nasal insertion probe is coupled to the stimulator body.

11. The nasal insertion probe of claim 1, further comprising:
a second extension of a second nasal insertion prong configured for insertion into a nasal cavity; and
a second electrode coupled to a distal end of the second extension and including the conductive plastic material.

12. A handheld stimulator system configured to provide a stimulation to nasal tissue of a subject, the handheld stimulator system comprising:
a stimulator body including a power source; and
a nasal stimulator probe configured to releasably couple to the stimulator body, the nasal stimulator probe comprising
a first extension of a first nasal insertion prong configured for insertion into a nasal cavity; and
a first electrode configured to provide the stimulation and coupled to a distal end of the first extension, the first electrode including a conductive plastic material.

13. A method of a handheld stimulator system, the method comprising:
delivering, via a conductive plastic material of a nasal stimulator probe of the handheld stimulator system, a stimulation to a nasal tissue of a subject, the nasal stimulator probe comprising
a first extension of a first nasal insertion prong configured for insertion into a nasal cavity; and
a first electrode configured to provide the stimulation and coupled to a distal end of the first extension, the first electrode including a conductive plastic material.

14. The method of claim 13, further comprising:
releasably coupling the nasal insertion probe to a stimulator body including a power source.

15. The method of claim 13, wherein the conductive plastic material includes a carbon black material.

16. The method of claim 13, wherein the conductive plastic material includes one or more of a graphene material, carbon fibers, and a metal polymer.

17. The method of claim 13, wherein the conductive plastic material assists with providing a conductive pathway between the power source of the stimulator body and nasal tissue.

18. The method of claim 13, wherein the conductive plastic material includes one or more of a polyethylene material, an ethylene vinyl acetate material, and a polypropylene material.

19. The method of claim 13, wherein the conductive plastic material of the first electrode includes a volume comprising approximately 3% to approximately 30% carbon black filler.

20. The method of claim 13, wherein the nasal stimulator probe further comprises
a second extension of a second nasal insertion prong configured for insertion into a nasal cavity; and
a second electrode coupled to a distal end of the second extension and including the conductive plastic material.
